(19) United States
(12) Patent Application Publication
(10) Pub. No.: US 2010/0256505 A1
(43) Pub. Date: Oct. 7, 2010

(54) Health monitoring method and system

(76) Inventors: Jingping Xu, Vancouver, WA (US); Deepak Ayyagari, Potomac, MD (US); Yongji Fu, Aloha, OR (US)

Correspondence Address: Scott A. Reader, Suite 228, 1320 Pearl Street, Boulder, CO, 80302, US

(21) Appl. No.: 12/384,367

(22) Filed: Apr. 3, 2009

(51) Int. Cl. A61B 5/0205

(52) U.S. Cl. 600/484

(57) ABSTRACT

A heath monitoring method and system estimate a patient's respiratory rate and heart rate using different frequency components of a shared acoustic signal. Use of a common acoustic signal to estimate the patient's respiratory rate and heart rate permit more economical and simplified heath monitoring.

### BACKGROUND OF INVENTION

This invention relates to health monitoring and, more particularly, to a health monitoring method and system that determine a patient's respiratory rate and heart rate in a more economical and simplified manner. The invention is especially useful as a portable system in ambulatory monitoring applications.

Respiratory rate and heart rate are important parameters used in monitoring the health status of patients in critical care facilities and in ambulatory monitoring of patients with chronic diseases, such as asthma. In conventional health monitoring systems, these two key parameters are estimated and outputted by systems that employ different data capture techniques and operate wholly independently of one another.

Several different systems may be used to estimate a patient's respiratory rate. Some respiratory rate estimation systems are airflow systems. In an airflow system, the patient breathes into an apparatus that measures the airflow through his or her mouth and the patient's respiratory rate is estimated from the airflow. Other systems measure the patient's volume, movement or tissue concentrations. For example, in a respiratory inductance plethysmography (RIP) system, the patient wears a first inductance band around his or her ribcage and a second inductance band around his or her abdomen. As the patient breathes, the volumes of the ribcage and abdominal compartments change, which alter the inductance of coils, and the patient's respiratory rate is estimated based on the changes in inductance. Still other systems are lung sound systems. In a lung sound system, an acoustic transducer generates an acoustic signal from which the patient's respiratory rate is estimated.

The systems used to estimate a patient's heart rate are different than those used to estimate a patient's respiratory rate. One heart rate estimation system known as a pulse oximeter (SpO2) utilizes optical sensing. In a SpO2 system, the patient's pulse rate is estimated based on the oxygen saturation in his or her blood as measured by oxygenated and deoxygenated haemoglobin. Other systems measure heart rate based on an electrocardiograph (ECG) signal. Other systems count carotid arterial pulse or pulse in other places. There are also systems that estimate heart rate using heart sounds detected at positions of the body, such as the trachea and chest.

Reliance on systems that use different data capture techniques and operate wholly independently of one another to estimate and output a patient's respiratory rate and heart rate adds component and interfacing costs and complexity to health monitoring systems.

### SUMMARY OF THE INVENTION

The present invention, in a basic feature, provides a heath monitoring method and system that estimate a patient's respiratory rate and heart rate using different frequency components of a shared acoustic signal acquired from the body. Use of a common acoustic signal to estimate the patient's respiratory rate and heart rate permit more economical and simplified heath monitoring.

In one aspect of the invention, a health monitoring system comprises an acoustic transducer, a signal processor communicatively coupled with the acoustic transducer and an output interface communicatively coupled with the signal processor, wherein the signal processor receives an acoustic signal based on sound detected by the acoustic transducer, generates respiratory rate data using a first frequency component of the acoustic signal, generates heart rate data using a second frequency component of the acoustic signal and transmits the respiratory rate data and the heart rate data to the output interface.

In some embodiments, the output interface comprises a user interface on which the respiratory rate data and heart rate data are displayed.

In some embodiments, the first frequency component comprises an approximation of a respiratory sequence.

In some embodiments, the signal processor isolates the first frequency component by applying a band-pass filter to the acoustic signal.

In some embodiments, the signal processor determines the respiratory rate data using a peak analysis of an autocorrelated envelope for the first frequency component.

In some embodiments, the second frequency component comprises an approximation of a pulse sequence.

In some embodiments, the signal processor isolates the second frequency component by applying a band-pass filter to the acoustic signal.

In some embodiments, the signal processor determines the heart rate data using a peak analysis of an autocorrelated envelope for the second frequency component.

In some embodiments, the respiratory rate data comprise an average respiratory rate and the heart rate data comprise an average heart rate.

In some embodiments, the signal processor transmits the respiratory rate data and the heart rate data to the output interface in real-time.

In another aspect of the invention, a health monitoring method comprises the steps of generating an acoustic signal based on detected sound, generating respiratory rate data using a first frequency component of the acoustic signal, generating pulse rate data using a second frequency component of the acoustic signal and outputting the respiratory rate data and the pulse rate data.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a health monitoring system in some embodiments of the invention.

FIG. 2 shows steps of a heath monitoring method performed by respiratory rate logic to generate respiratory rate data in some embodiments of the invention.

FIG. 3 shows steps of a health monitoring method performed by heart rate logic to generate heart rate data in some embodiments of the invention.

FIG. 4 shows an exemplary raw acoustic signal.

FIG. 5 shows an exemplary acoustic signal after application of a band-pass filter to the signal of FIG. 4.

FIG. 6 shows an exemplary acoustic signal envelope after application of an envelope detector and smoothing module to the signal of FIG. 5.

FIG. 7 shows an exemplary acoustic signal envelope after application of an autocorrelation module to the signal of FIG. 6.

FIG. 8 shows an exemplary acoustic signal after application of a band-pass filter to the signal of FIG. 4.

FIG. 9 shows an exemplary acoustic signal envelope after application of an envelope detector and smoothing module to the signal of FIG. 8.

FIG. 10 shows an exemplary acoustic signal envelope after application of an autocorrelation module to the signal of FIG. 9.

### DETAILED DESCRIPTION OF A PREFERRED EMBODIMENT

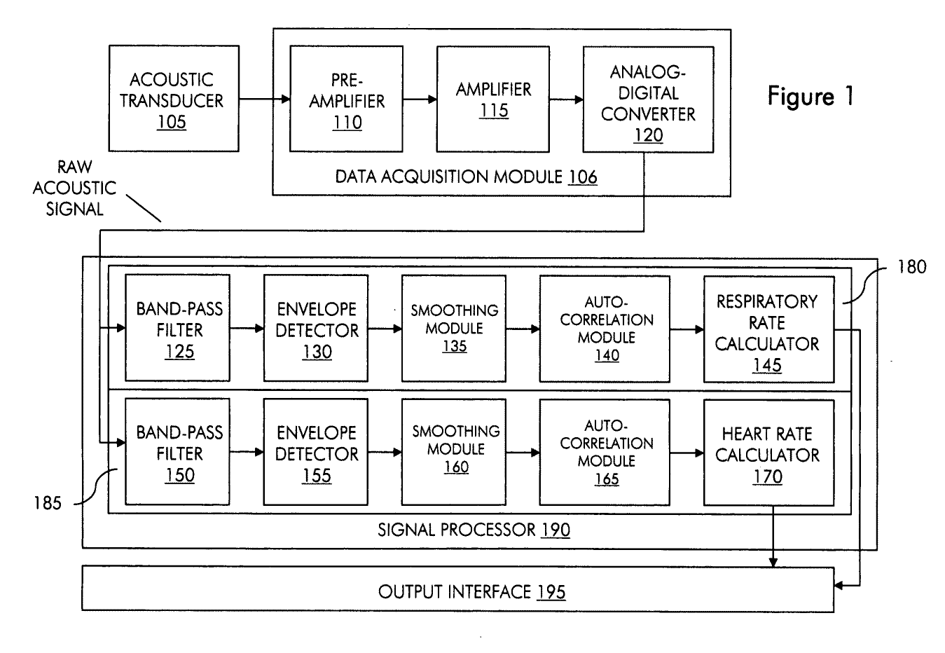

FIG. 1 shows a health monitoring system in some embodiments of the invention. The system includes an acoustic transducer 105 positioned on the body of a patient who is being monitored. Transducer 105 is communicatively coupled in series with data acquisition module 106 that includes a pre-amplifier 110, amplifier 115 and an analog-to-digital (A/D) converter 120. A/D converter 120 continually transmits a raw acoustic signal collected from transducer 105, as modified by amplifiers 110, 115, to a signal processor 190. Signal processor 190 continually generates respiratory rate data and heart rate data using different frequency components of the raw acoustic signal and continually transmits the respiratory rate data and heart rate data to an output interface 195. While elements 110-120 are shown collocated on data acquisition module 106 and elements 125-170 are shown collocated on signal processor 190, in other embodiments elements shown in FIG. 1 may be collocated with different elements shown in FIG. 1 or may be stand-alone elements. Moreover, elements that are not collocated may be located in proximity to or remotely from one another and may be communicatively coupled via wired or wireless connections. In some embodiments, signal processor 190 and output interface 195 are collocated on a mobile electronic device. In these embodiments, the device may be attached to the patient's clothing (e.g. clipped-on), or a handheld device that is carried by the patient, for example. Moreover, in some embodiments the respiratory rate data and heart rate data may be outputted to multiple output interfaces.

Transducer 105 detects sound at a position on the patient's body, such as the trachea or chest. Transducer 105 provides high sensitivity, a high signal-to-noise ratio and a generally flat frequency response in the band for lung sounds. Transducer 105 in some embodiments comprises an omni-directional piezo ceramic microphone housed in an air chamber of suitable depth and diameter. A microphone marketed by Knowles Acoustics as part BL-21785 may be used by way of example. Transducer 105 outputs to data acquisition module 106 a raw acoustic signal based on detected sound to pre-amplifier 110 as an analog voltage on the order of 10-200 mV.

At data acquisition module 106, pre-amplifier 110 provides impedance match for the raw acoustic signal received from transducer 105 and amplifies the raw acoustic signal. A pre-amplifier marketed by Presonus Audio Electronics as TubePre Single Channel Microphone Preamp with VU (Volume Unit) Meter may be used by way of example.

Amplifier 115 further amplifies the raw acoustic signal received from amplifier 110 to the range of +/−1 V.

A/D converter 120 performs A/D conversion on the raw acoustic signal received from amplifier 115 and transmits the raw acoustic signal to signal processor 190 for analysis.

Signal processor 190 is a microprocessor having software executable thereon for performing signal processing on the raw acoustic signal received from data acquisition module 106. At signal processor 190, the raw acoustic signal is split and the dual instances of the raw acoustic signal are processed by respiratory rate logic 180 and heart rate logic 185, respectively, to generate and transmit to output interface 195 in real-time an average respiratory rate and average heart rate, respectively. In other embodiments, all or part of the functions of signal processor 190 may be performed in custom logic, such as one or more application specific integrated circuits (ASIC).

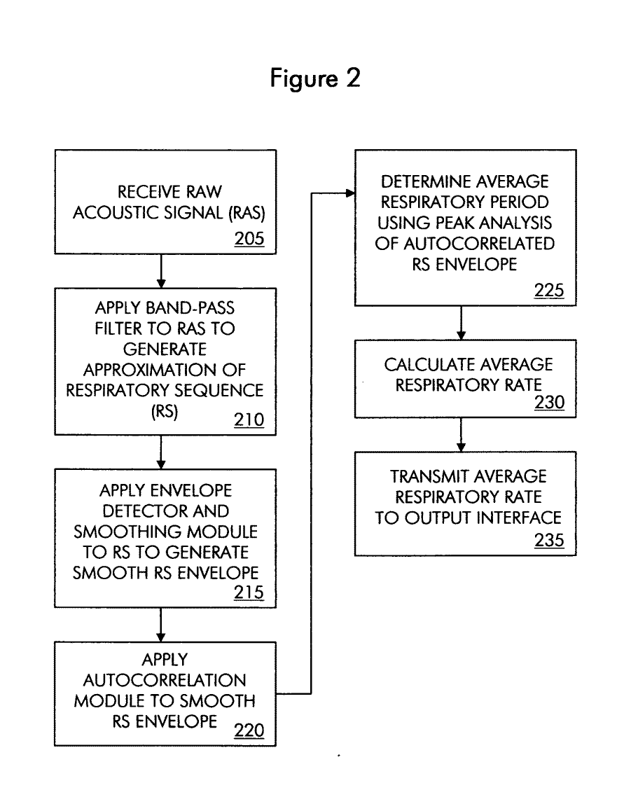

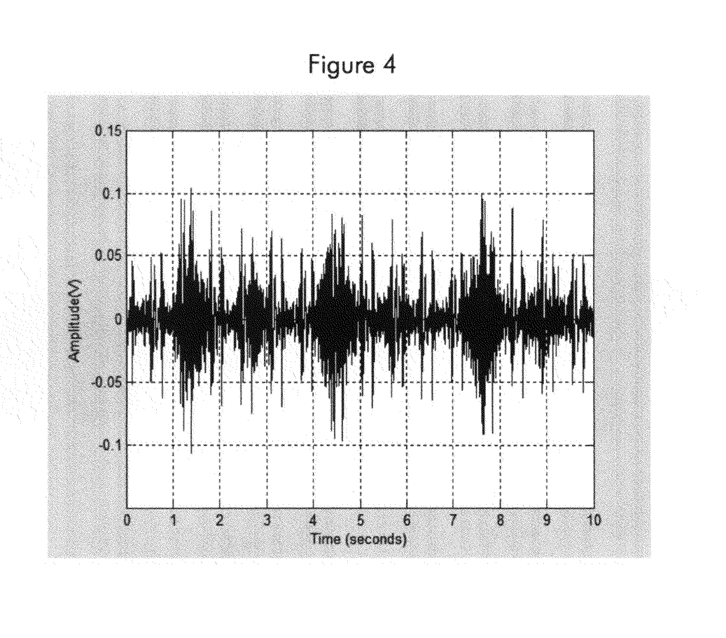

Respiratory rate logic 180 includes a band-pass filter 125, an envelope detector 130, a smoothing module 135, an autocorrelation module 140 and a respiratory rate calculator 145. Steps of a health monitoring method performed by respiratory rate logic 180 to generate respiratory rate data in some embodiments of the invention are shown in FIG. 2 and will be described by reference to FIGS. 4-7.

Initially, the raw acoustic signal is received (205) from data acquisition module 106. An exemplary raw acoustic signal is shown in FIG. 4. The raw acoustic signal is noisy and the pulse sequence is intermingled with the respiratory sequence.

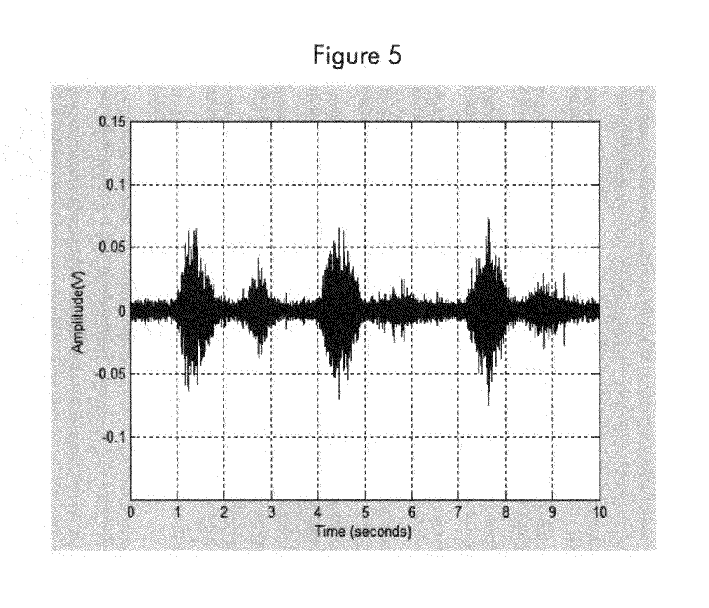

Next, band-pass filter 125 applies a high-pass cutoff frequency at 100 Hz and a low-pass cutoff frequency at 900 Hz to the acoustic signal to isolate a first frequency component of the signal that approximates the respiratory sequence (RS) (210). An exemplary resulting signal is shown in FIG. 5. The pulse sequence has been removed and the respiratory sequence is better defined due to noise reduction.

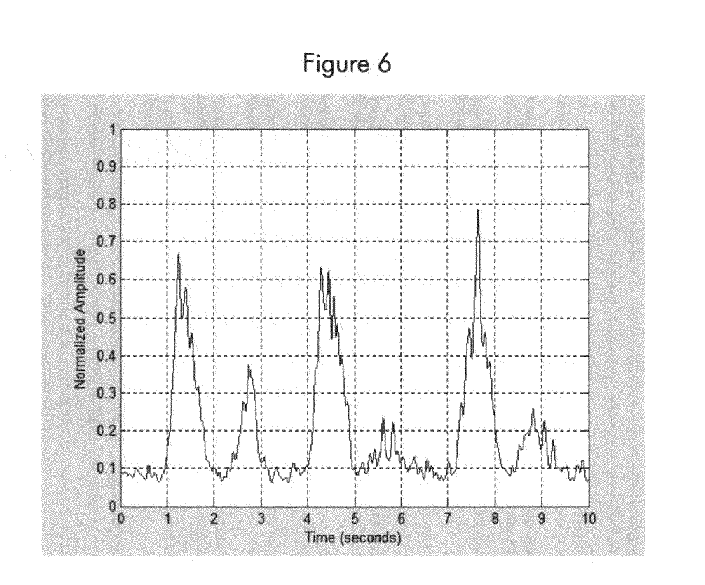

Next, an envelope detector 130 and smoothing module 135 are applied to the RS acoustic signal to generate a smooth RS envelope (215). Smoothing module 135 removes additional noise from the RS acoustic signal and improves signal quality. In some embodiments, smoothing module 135 applies to the RS acoustic signal a smooth FIR filter with order in the range of 800 to 1200 [e.g. a Hanning (Hann) window with order of 1000]. An exemplary resulting smooth RS envelope is shown in FIG. 6.

In some embodiments, at this point a down-sampler (not shown) down-samples the smooth RS envelope to a lower sampling frequency in order to reduce the sampled data length and save computational resources.

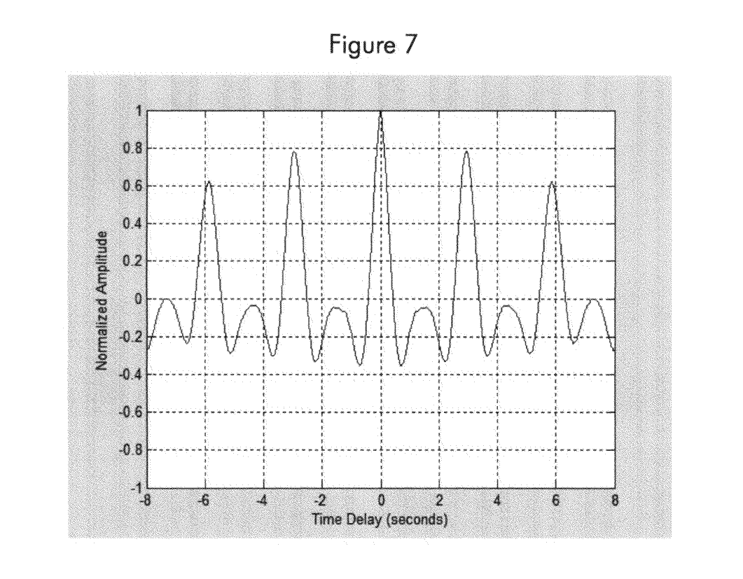

Next, autocorrelation module 140 is applied to the smooth RS envelope to identify the fundamental periodicity of the data (220). An exemplary resulting autocorrelated smooth RS envelope is shown in FIG. 7. There is a maximum peak at zero time delay. The time distance to the adjacent peak of similar amplitude in either direction corresponds to the average respiratory period across multiple cycles.

Next, respiratory rate calculator 145 determines an average respiratory period using peak analysis of the autocorrelated smooth RS envelope (225). The average respiratory period is identified as the peak-to-peak time difference between the highest peak and the next peak of similar amplitude in the positive or negative direction within the autocorrelated smooth RS envelope. In the example shown in FIG. 7, the time difference between the highest peak and the next peak of similar amplitude in the positive direction is 2.958 seconds, which may be identified and applied as the average respiratory period.

Next, respiratory rate calculator 145 determines an average respiratory rate based on the average respiratory period (230). The average respiratory rate in breaths per minute is 60 divided by the average respiratory period. Returning to the example shown in FIG. 7, the average respiratory rate is 60/2.958 or 20.284 breaths per minute.

Finally, signal processor 190 transmits the average respiratory rate to output interface 195 (235). In some embodiments, output interface 195 is a user interface that displays the average respiratory rate data to the patient in real-time. In other embodiments, output interface 195 is a computing system that further processes the respiratory rate data.

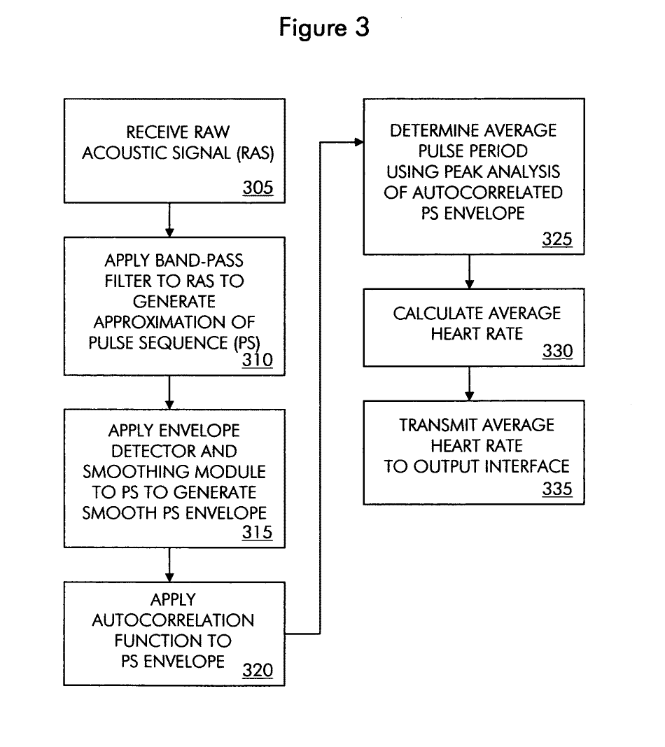

Heart rate logic 185 includes a band-pass filter 150, an envelope detector 155, a smoothing module 160, an autocorrelation module 165 and a heart rate calculator 170. Steps of a health monitoring method performed by heart rate logic 185 to generate heart rate data in some embodiments of the invention are shown in FIG. 3 and will be described by reference to FIGS. 4 and 8-10.

Initially, the raw acoustic signal is received (305) from data acquisition module 106. An exemplary raw acoustic signal is shown in FIG. 4. The raw acoustic signal is noisy and the respiratory sequence is intermingled with the pulse sequence.

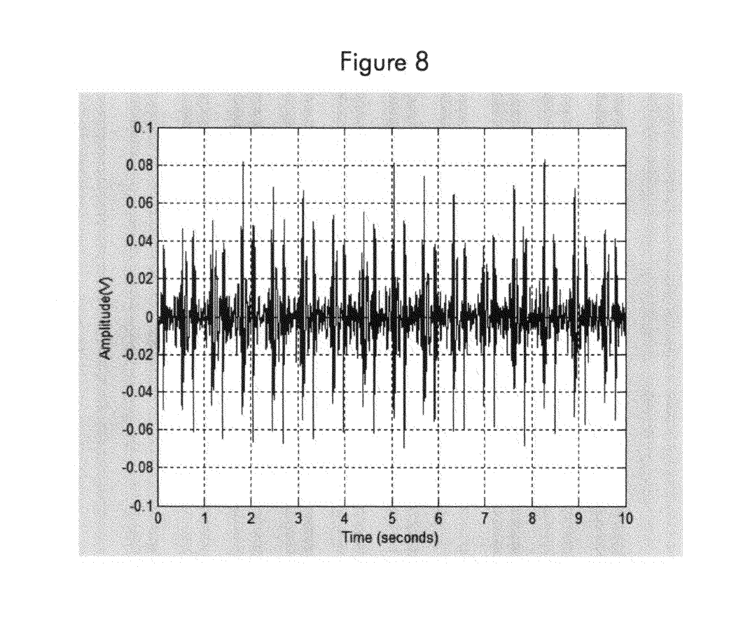

Next, band-pass filter 150 applies a cutoff frequency at 100 Hz to the acoustic signal to isolate a second frequency component of the signal that approximates the pulse sequence (PS) (310). An exemplary resulting signal is shown in FIG. 8. The respiratory sequence has been removed and the pulse sequence is better defined due to noise reduction.

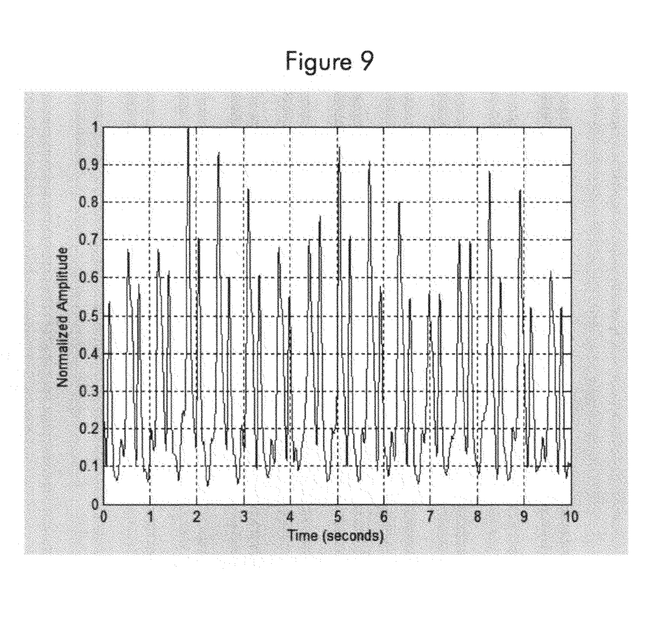

Next, an envelope detector 155 and smoothing module 160 are applied to the PS acoustic signal to generate a smooth PS envelope (315). Smoothing module 160 removes additional noise from the PS acoustic signal and improves signal quality. In some embodiments, smoothing module 160 applies to the PS acoustic signal a smooth FIR filter with order in the range of 800 to 1200 [e.g. a Hanning (Hann) window with order of 1000]. An exemplary resulting smooth PS envelope is shown in FIG. 9.

At this point a down-sampler may down-sample the PS envelope to a lower sampling frequency in order to reduce the sampled data length and save computational resources.

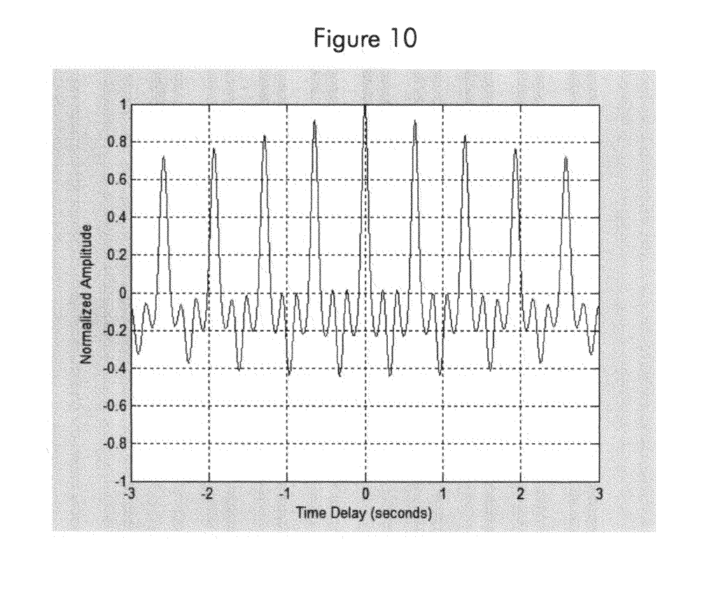

Next, autocorrelation module 165 is applied to the smooth PS envelope to identify the fundamental periodicity of the data (320). An exemplary resulting smooth autocorrelated PS envelope is shown in FIG. 10. There is a maximum peak at zero time delay. The time distance to the adjacent peak of similar amplitude in either direction corresponds to the average pulse period across multiple cycles.

Next, heart rate calculator 170 determines an average pulse period using peak analysis of the smooth autocorrelated PS envelope (325). The average pulse period is identified as the peak-to-peak time difference between the highest peak and the next peak of similar amplitude in the positive or negative direction within the smooth autocorrelated PS envelope. In the example shown in FIG. 10, the time difference between the highest peak and the next peak of similar amplitude in the positive direction is 0.6463 seconds, which may be identified and applied as the average pulse period.

Next, heart rate calculator 170 determines an average heart rate based on the average pulse period (330). The overage heart rate in beats per minute is 60 divided by the average pulse period. Returning to the example shown in FIG. 10, the average heart rate is 60/0.6463 or 92.836 beats per minute.

Finally, signal processor 190 transmits the average heart rate to output interface 195 (335) for further processing and/or display.

In some embodiments, output interface 195 is a user interface. In these embodiments, output interface 195 may be a liquid crystal display (LCD) or light emitting diode (LED) panel that displays the most recent average respiratory rate and average heart rate to the patient. Since the current respiratory rate data and heart rate data are generated from a shared acoustic signal and outputted on the same user interface at approximately same time, interfacing and synchronization complexities are avoided.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A health monitoring system, comprising:
an acoustic transducer;
a signal processor communicatively coupled with the acoustic transducer; and
an output interface communicatively coupled with the signal processor, wherein the signal processor receives an acoustic signal based on sound detected by the acoustic transducer, generates respiratory rate data using a first frequency component of the acoustic signal, generates heart rate data using a second frequency component of the acoustic signal and transmits the respiratory rate data and the heart rate data to the output interface.

2. The system of claim 1, wherein the output interface comprises a user interface on which the respiratory rate data and the heart rate data are displayed.

3. The system of claim 1, wherein the first frequency component comprises an approximation of respiratory sequence.

4. The system of claim 1, wherein the signal processor isolates the first frequency component by applying a band-pass filter to the acoustic signal.

5. The system of claim 1, wherein the signal processor determines the respiratory rate data using a peak analysis of an autocorrelated envelope for the first frequency component.

6. The system of claim 1, wherein the second frequency component comprises an approximation of pulse sequence.

7. The system of claim 1, wherein the signal processor isolates the second frequency component by applying a band-pass filter to the acoustic signal.

8. The system of claim 1, wherein the signal processor determines the heart rate data using a peak analysis of an autocorrelated envelope for the second frequency component.

9. The system of claim 1 wherein the respiratory rate data comprise an average respiratory rate and the heart rate data comprise an average heart rate.

10. The system of claim 1 wherein the signal processor transmits the respiratory rate data and the heart rate data to the output interface in real-time.

11. A health monitoring method, comprising the steps of:
generating an acoustic signal based on detected sound;
generating respiratory rate data using a first frequency component of the acoustic signal;
generating heart rate data using a second frequency component of the acoustic signal; and
outputting the respiratory rate data and the heart rate data.

12. The method of claim 11, wherein the outputting step comprises displaying the respiratory rate data and the heart rate data on a user interface.

13. The method of claim 11, wherein the first frequency component comprises an approximation of respiratory sequence.

14. The method of claim 11, wherein the first frequency component is isolated by applying a band-pass filter to the acoustic signal.

15. The method of claim 11, wherein the respiratory rate data are determined using a peak analysis of an autocorrelated envelope for the first frequency component.

16. The method of claim 11, wherein the second frequency component comprises an approximation of pulse sequence.

17. The method of claim 11, wherein the second frequency component is isolated by applying a band-pass filter to the acoustic signal.

18. The method of claim 11, wherein the pulse rate data are determined using a peak analysis of an autocorrelated envelope for the second frequency component.

19. The method of claim 11, wherein the respiratory rate data comprise an average respiratory rate and the heart rate data comprise an average heart rate.

20. The method of claim 11, wherein the outputting step is performed in real-time.
